(19) United States
(12) Patent Application Publication
(10) Pub. No.: US 2025/0002427 A1
(43) Pub. Date: Jan. 2, 2025

(54) PROCESS FOR CONVERTING PLASTIC WASTE INTO BASE CHEMICALS

(71) Applicant: Charlie Caviel, III, Houston, TX (US)

(72) Inventor: Charlie Caviel, III, Houston, TX (US)

(21) Appl. No.: 18/757,359

(22) Filed: Jun. 27, 2024

Related U.S. Application Data
(60) Provisional application No. 63/523,545, filed on Jun. 27, 2023.

(51) Int. Cl. C07C 4/22; C08J 11/20; C10G 1/10

(52) U.S. Cl. CPC C07C 4/22; C08J 11/20; C10G 1/10; C08J 2323/06; C08J 2323/12; C10G 2300/1003; C10G 2400/20; C10G 2400/22; C10G 2400/30

(57) ABSTRACT

This invention provides an advanced process for converting plastic waste into base chemicals. In various embodiments, the process includes one or more of the following process steps, including cleaning, shredding, dissolving plastic waste in an aromatic solvent, pre-fractionation, catalytic decomposition, fractionation, and coking. This method effectively breaks down plastic waste into valuable chemicals and high-quality reusable plastic.

### CROSS REFERENCE TO RELATED APPLICATIONS

This application claims the benefit of U.S. Provisional Patent Application No. 63/523,545, filed Jun. 27, 2023, the entire contents of which is hereby incorporated by reference.

### BACKGROUND OF THE INVENTION

Plastic waste accumulation is a pressing issue with that has far-reaching environmental, health, and socio-economic implications. Each year, vast amounts of plastic materials are produced worldwide, a significant portion of which ends up as waste. This waste often doesn't decompose for hundreds of years, leading to persistent pollution that harms wildlife, contaminates ecosystems, and contributes to global climate change. Moreover, plastic waste can clog urban infrastructure, leading to sanitation issues, and when improperly incinerated, can release harmful toxins. Additionally, the current linear economy model of “make, use, dispose” results in a tremendous loss of potential resources, as valuable materials are discarded after single use.

A number of plastic waste recovery processes are known.

One example involves the thermal treatment or decomposition of plastic waste in the absence of oxygen (a process known as pyrolysis). The process can convert plastic waste into a variety of useful products, including pyrolysis oil (which can be used as a fuel or as a feedstock for producing new plastics), syngas, and char. However, the quality of the products can vary depending on the type of plastic waste and the specific conditions of the pyrolysis process.

Pyrolysis and other known plastic waste recovery processes suffer from a number of limitations, including energy inefficiency, limited applicability to different types of plastic wastes, poor product quality, and production of environmental pollutants.

Therefore, a need persists for plastic waste recovery techniques and processes capable of effectively and efficiently breaking down plastic waste into its base molecules of suitable purity for manufacture of new plastic products and other uses. Such a process will reduce the need for virgin plastic production and effectively remove contaminants from the environment, promoting a sustainable circular economy.

### SUMMARY OF THE INVENTION

The present invent relates to processes for the recovery of base chemicals from plastic waste.

In accordance with one embodiment, the process comprises reducing the plastic waste to produce a particulate plastic waste; dissolving the particulate plastic waste in an aromatic solvent to produce a dissolved plastic solution effluent; fractionating the dissolved plastic solution effluent to produce an overheads fraction and a bottoms fraction; catalytically decomposing the components of the bottom fraction to produce decomposition products; and subjecting at least a portion of the decomposition products to a coking operation.

Other features, objects and advantages of the invention will be in part apparent and in part pointed out hereinafter in the description and claims.

### BRIEF DESCRIPTION OF THE DRAWINGS

FIG. 1 is a schematic drawing of the process for recovery of base chemicals from plastic waste.

### DETAILED DESCRIPTION OF THE INVENTION

The present invention involves a series of steps to convert plastic waste into base chemicals utilizing catalytic reactions and delayed coking operations. This process effectively removes contaminants and produces high-purity end products.

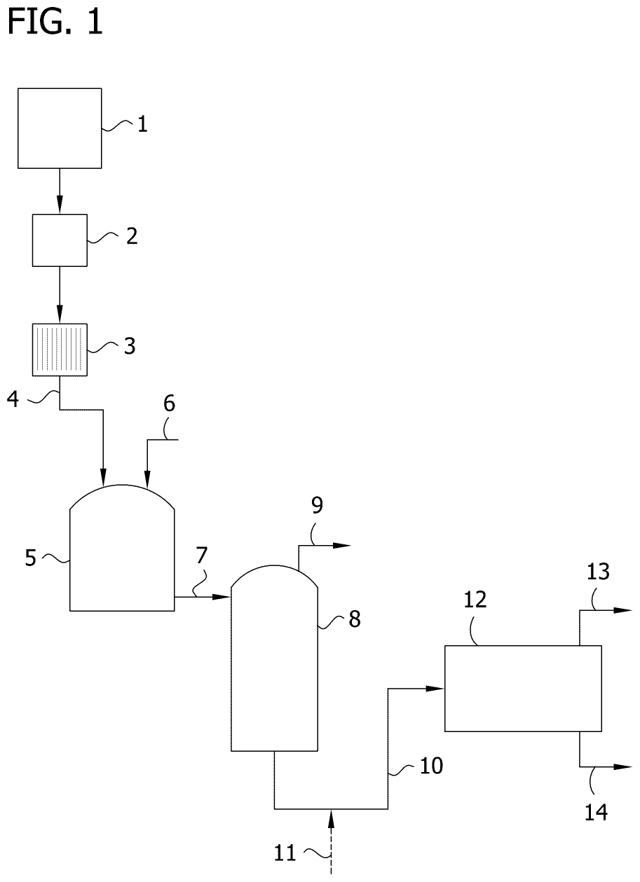

FIG. 1 is a is a schematic drawing of the process for recovery of base chemicals from plastic waste.

As described in greater detail below, the process includes collected plastic waste 1, which may be subjected to optional cleaning operations 2 before or after particle size reduction/shredding operation 3, to produce a particulate plastic waste 4 from which useful base molecules are recovered.

Particulate plastic waste 4 is directed to a dissolution unit 5 and combined with a dissolution medium 6 comprising an aromatic solvent and heated therein to produce a dissolved plastic solution effluent 7 comprising dissolved plastic molecules. Following recovery of the aromatic solvent, dissolved plastic solution effluent 7 is separated into its constituent components based on boiling points in a pre-fractionation operation 8 to produce an overheads fraction 9 comprising lighter compounds/base molecules for recovery and a bottoms fraction 10 comprising heavier compounds/base molecules for recovery. Bottoms fraction 10 from pre-fractionation operation 8 is directed to catalytic decomposition/coking operation in coker unit 12 that produces vaporized light hydrocarbons 13 for recovery as valuable products and coke bottoms 14.

### Plastic Waste

Plastic waste 1 treated in accordance with the invention may include both post-consumer and pre-consumer plastic waste and can be collected from municipal waste streams, industrial waste, and commercial waste. Collection methods include curbside recycling programs, drop-off centers, and direct collection from manufacturing facilities.

Various types of plastic materials may be included in the plastic waste directed to the process and include, without limitation, high-density polyethylene (HDPE), low-density polyethylene (LDPE), linear low-density polyethylene (LLDPE), polypropylene (PP), polyethylene terephthalate (PET) and mixtures thereof. These materials and other common types of plastic materials present in plastic waste streams are primarily composed of long chains of ethylene, propylene and other monomers, making them suitable for recovery of base chemicals therefrom in accordance with the present invention.

### Cleaning Operations

Depending on the sources from which it is collected (e.g., post-consumer), the plastic waste may be contaminated with food and industrial residues, labels, adhesives, and dirt. Accordingly, the process of the present invention optionally includes cleaning the plastic waste in order to avoid negative effects during subsequent processing and enhance the quality and purity of the products recovered from the process.

In FIG. 1, cleaning operations 2 may include separation tanks filled with water into which the incoming mixture of plastic waste and contaminants is directed to separate plastics from other materials based on density differences. Once separated, high-pressure water jets can be used for initial cleaning of the separated plastic waste followed by contacting with a solution of water and detergent. Brushes, rollers and agitators can optionally be used to physically remove contaminants from the plastic waste as part of the cleaning operations. The cleaned plastic waste may optionally be rinsed with water and dried (e.g., using air dryers or centrifugal drying systems) to remove moisture.

### Particle Size Reduction/Shredding

As shown in FIG. 1, the collected and optionally cleaned plastic waste is shredded or subjected to other particle size reduction operation 3 to produce particulate plastic waste 4. Particle size reduction increases the specific surface area and prepares the waste for more efficient subsequent processing.

The equipment used to reduce the collected plastic waste may be selected from industrial shredders, granulators, grinders and other high-capacity machines with rotating blades or other cutting tools to cut the plastic waste into small pieces. Typically, the plastic waste is reduced such that it exhibits an average particle size of from about 10 mm to about 50 mm in largest dimension. Shredding or other particle size reduction ensures uniform particle sizes, which enhances the efficiency of subsequent processing steps.

In accordance with some embodiments of the present invention, the collected plastic waste may alternatively be subjected to particle size reduction prior to cleaning operations.

The recovery of useful base molecules from the particulate plastic waste 4 can be initiated in various ways.

### Dissolution in an Aromatic Solvent

In accordance with one embodiment, the particulate plastic waste 4 is directed to dissolution unit 5 and combined with dissolution medium 6 comprising an aromatic solvent. The resulting mixture is heated in the dissolution unit to produce a dissolved plastic solution effluent 7 comprising dissolved plastic molecules.

Aromatic solvents used in dissolving plastic waste typically exhibit a specific gravity of about 0.86 to about 0.91 to ensure efficient dissolution and have a molecular weight of at least about 78 to 106 g/mol to maintain solvent stability. For safe handling, the aromatic solvent desirably exhibits a flash point above about 15° C. (59° F.). Examples of suitable aromatic solvents include toluene, xylene, benzene, styrene, ethylbenzene, and mixtures thereof.

The dissolution unit 5 suitably comprises a heated stirred tank equipped with an impeller or similar mixing apparatus or a homogenizer to ensure uniform dissolution of the plastic particles into the dissolution medium. The shredded plastic particles 4 are fed into the dissolution unit containing the dissolution medium comprising the aromatic solvent, and the mixture is heated to the required temperature to produce dissolved plastic solution effluent 7. The temperature range for dissolution of plastics in aromatic solvents such as toluene or xylene is generally about 150° C. to about 300° C. The decomposition temperature range, where plastics begin to chemically break down, is typically 350° C. to 500° C.

A catalyst can be employed to enhance the dissolution of the plastic waste in the dissolution medium. When a catalyst is used, the presence of the catalyst can lower the temperature required for dissolving plastics in aromatic solvents. The suitable temperature range for dissolution with a catalyst is generally about 100° C. to about 200° C. However, the decomposition temperature range, where plastics begin to chemically break down, remains the same regardless of the presence of a catalyst (i.e., typically 350° C. to 500° C.).

In addition to the aromatic solvent (e.g., toluene or xylene), the dissolved plastic solution effluent will typically contain dissolved plastic molecules (i.e., polymers broken down into smaller units); impurities and contaminants (e.g., residual impurities from the plastic waste); additives (e.g., additives initially present in the plastic such as stabilizers and colorants) and potential decomposition products (e.g., minor amounts of degradation products depending on process conditions). Typically, the concentration of dissolved plastic molecules in the dissolved plastic solution effluent is from about 1 to about 30 wt. %.

### Solvent Recovery

The aromatic solvent used in the waste plastic dissolution operation can be usefully recovered by various methods. Although not shown in FIG. 1, solvent is typically recovered from the dissolved plastic solution effluent 7 after the dissolution of plastic and before fractionation, ensuring that the solvent, which is a valuable and reusable resource, is separated from the dissolved plastic solution efficiently. The primary reason for recovering the solvent before fractionation is to recycle and reuse the solvent, reducing operational costs and minimizing environmental impact.

Distillation: The solvent is heated to its boiling point, vaporized, and then condensed back to a liquid form. This method is effective in separating the solvent from the dissolved plastic.

Membrane Separation: A selective membrane is used to separate the solvent from the plastic solution based on size or chemical affinity, which is efficient for continuous operations.

Adsorption: Adsorbent materials like activated carbon capture the solvent molecules, which is useful for solutions with lower solvent concentrations or for polishing the solvent to high purity levels.

After solvent recovery, what remains is a concentrated plastic solution, which contains the dissolved polymers and any remaining impurities. This concentrated solution then undergoes fractionation to separate the components based on their boiling points. The fractionation process yields different products such as monomers, oligomers, or other useful chemicals that can be further processed or used.

### Pre-Fractionation/Initial Separation

The dissolved plastic solution effluent 7 (following optional solvent recovery therefrom) is separated into its constituent components based on boiling points. In this pre-fractionation operation 8, the dissolved plastic solution effluent is fed to a suitable distillation apparatus, such as a vertical tray or plate column or a packed column, to facilitate separation into its various components. Heating elements vaporize the solution, and components are separated based on their boiling points. The lighter fractions are collected at the top of the column as overheads fraction 9, while heavier fractions comprising heavier compounds/base molecules remain at the bottom and are recovered in bottoms fraction 10.

The compounds/base molecules present in the lighter fractions recovered in overheads fraction 9 typically include monomers (such as ethylene C₂H₄, molecular weight 28 g/mol; specific gravity 0.00126 at 25° C. derived from polyethylene; propylene C₃H₆, molecular weight 42 g/mol; specific gravity 0.00181 at 25° C. derived from polypropylene; and styrene C₈H₈, molecular weight 104 g/mol; specific gravity 0.906 at 20° C. derived from polystyrene), oligomers (such as short-chain hydrocarbons C₄-C₁₀ alkanes and alkenes, including butane C₄H₁₀, pentane C₅H₁₂, and hexane C₆H₁₄ and aromatics (such as benzene C₆H₆, molecular weight 78 g/mol; specific gravity 0.876 at 20° C.; toluene C₇H₈, molecular weight 92 g/mol; specific gravity 0.867 at 20° C.; and xylene C₈H₁₀, molecular weight 106 g/mol; specific gravity 0.86-0.88 at 20° C.).

During the fractionation process, at least a portion of the aromatic solvent can be recovered alongside the lighter fractions. The aromatic solvents, due to their boiling points, may co-distill with other light hydrocarbons and monomers. The aromatic solvent can be separated from the recovered base molecules by various means as noted above, including distillation (during which the mixture of aromatic solvent and lighter fractions is heated; the lighter components, including monomers and low-boiling oligomers, are vaporized and separated first; and the aromatic solvent, having a distinct boiling point, can then be condensed separately), membrane separation (a membrane allows certain molecules to pass through while retaining others based on size or chemical affinity to recover aromatic solvents from a mixture with lighter fractions), and adsorption (based on the affinity of the adsorbent material (e.g., activated carbon) for specific compounds captures the aromatic solvent molecules from the mixture resulting in separation of aromatic solvents from other lighter hydrocarbons and monomers).

In accordance with one particular embodiment, the plastic waste is dissolved in an aromatic solvent to form the dissolved plastic solution effluent 7. The initial solvent recovery step might use distillation to remove the bulk of the aromatic solvent for reuse, leaving a concentrated solution. The concentrated solution undergoes fractionation, separating components based on boiling points, with lighter fractions including some aromatic solvent being collected. The collected lighter fractions are subjected to additional distillation to separate the aromatic solvent from recovered monomers and other hydrocarbons. Alternatively, membrane separation or adsorption methods are employed to recover high-purity aromatic solvents. Recovering the aromatic solvent before fractionation is essential to recycle and reuse the solvent efficiently. The remaining solution, after solvent recovery, is then processed to extract valuable chemical components through fractionation.

### Catalytic Decomposition, Fractionation and Coking of Bottom Fraction

In accordance with some embodiments, bottoms fraction 10 produced in the pre-fractionation operation may be subjected to catalytic decomposition to breakdown these heavier components into smaller molecules and subjected to further fractionation and coking to recover base molecules.

As explained above, after the initial dissolution and pre-fractionation operations, the lighter fractions (such as monomers and light hydrocarbons) are separated, leaving the heavier residue as bottoms fraction 10. In accordance with one embodiment, this bottom fraction is fed into the coker unit 12 for further processing.

Bottoms fraction 10 delivered to the coker unit 12 consists of the heavier hydrocarbons that remain after the initial thermal processing and pre-fractionation steps. The bottoms fraction primarily includes heavy hydrocarbons, residual polymers, and other high-molecular-weight compounds that did not vaporize during the initial fractionation. These compounds are generally higher boiling point components and require further processing to break down into more valuable products.

In accordance with one embodiment, extruded plastic waste may optionally be combined with bottoms fraction 10 fed to coker unit 12. In such an embodiment, at least a portion of the shredded plastic and dissolution medium comprising an aromatic solvent are fed into an extruder. The extruder heats and mixes the materials, ensuring uniform dissolution. Various types of extruders may be employed, including: single-screw, twin-screw, planetary roller extruders. Single-screw extruders are suitable moderate throughput operations. Twin-screw extruders provide better mixing and higher throughput. Planetary roller extruders provide precise control and high shear mixing. Using advanced extruder technology to facilitate the dissolution process ensures uniform mixing and optimal dissolution conditions, leading to higher efficiency and quality. As shown in FIG. 1, the extruded plastic waste 11 is combined with bottoms fraction 10 fed to coker unit 12.

The catalytic decomposition can be carried out using various types of catalyst, including zeolite catalysts, metal oxide catalysts, and mixed metal catalysts. Zeolite catalysts are known for their high surface area and pore structure, which enhances reaction rates. Metal oxide catalysts such as aluminum oxide and titanium oxide, are characterized by their stability and activity. Mixed metal catalyst comprise a combinations of different metals to optimize catalytic activity. Those skilled in the art can readily select the type of catalyst and its composition based on the composition of the heavier bottom fraction subjected to decomposition.

The catalytic decomposition of the heavier bottoms fraction is conducted in a reactor designed to maintain and withstand the reaction conditions. In the embodiment shown in FIG. 1, the catalytic decomposition reaction is carried out in the coker unit 12 and is typically conducted at a temperature from about 300° C. to about 500° C., depending on the specific catalyst used. Using a combination of catalysts tailored to specific plastic types and operating within an optimal temperature range ensures efficient decomposition and high yield of base chemicals.

The effluent comprising the decomposition products exiting the reaction are directed to a further fractionation (distillation) to separate the decomposed products based on their boiling points into an overheads fraction and a bottoms fraction.

Delayed coking is a specific type of coking process that is widely used in the petroleum and petrochemical industries due to its efficiency and ability to handle heavy residues. In the practice of the present invention, existing coker units in petrochemical facilities can be adapted for processing plastic waste by adjusting feedstock handling and reactor conditions. Repurposing existing coker units for plastic waste processing enhances sustainability and integrates seamlessly into current petrochemical infrastructure.

A delayed coker operates in cycles, where feedstock is heated in a furnace and then coked in large drums. Delayed coking offers several process advantages. Delayed coking involves thermal cracking of heavy residues at high temperatures, breaking them down into lighter hydrocarbons and solid coke. Delayed coking effectively processes heavy residues, converting them into valuable lighter products like naphtha, diesel, and gas oils, as well as producing coke as a by-product. Delayed coker units can handle a wide range of feedstocks, including heavy fractions from plastic decomposition.

In accordance with some embodiments, other coker configurations may be employed, but are generally less advantageous. Various types of coker configurations known in the art can be used in the coking operation, including fluid cokers and flexicoker configurations. A flexicoker provides a flexible coking operation that can handle various feedstocks and produces coke and lighter hydrocarbons. Flexi-coking, although more efficient in converting heavy residues into lighter products, flexi-coking is more complex and expensive to operate. A fluid coker uses fluidized beds to enhance heat transfer and coke formation. Fluid coking is suitable for specific applications, but less common than delayed coking due to higher operational complexity and costs.

Accordingly, delayed coking is often preferred because it strikes a balance between operational efficiency, cost, and the ability to handle a variety of feedstocks. The feedstock to the delayed coking operation comprising heavy bottoms fraction 10, and optionally extruded plastic waste 11, is heated and fed into the coker drum. In the coker drum, thermal cracking occurs at temperatures typically between about 480° C. and about 510° C. (896° F. to 950° F.), breaking down the heavy hydrocarbons. This cracking produces light hydrocarbons that are vaporized and recovered as valuable products and coke, the solid carbonaceous material that is collected as a by-product. The recovered bottoms fraction following catalytic decomposition is directed to a delayed coking operation to convert the decomposed heavy fraction components into useful hydrocarbons and other chemical products.

The coking operation can produce a variety of useful products, including:

Aromatics: Benzene, toluene, xylene used in the chemical industry for producing solvents, dyes, and synthetic fibers.

Olefins: Ethylene, propylene used as building blocks for polyethylene and polypropylene production.

Naphtha: Used as a feedstock for producing high-octane gasoline components and petrochemicals.

Specialty Chemicals: Production of high-purity chemicals for pharmaceuticals, cosmetics, and industrial applications.

Furfural: A valuable chemical used in the production of resins, solvents, and as a precursor for various furan-based chemicals.

Coke: Used as a carbon source in steel manufacturing and as a fuel in industrial applications.

In accordance with one embodiment, wherein the objective is to break down plastic waste back into base chemicals using delayed coking, especially with a cleaner feedstock due to the absence of sulfur and other contaminants, the process must be precisely controlled.

In such an embodiment, the key parameters to control in the delayed coking operations include temperature in a range of from about 480° C. to about 510° C. (896° F. to 950° F.). High and consistent temperatures promote the breakdown of heavy hydrocarbons into lighter, valuable chemical intermediates like ethylene, propylene, and benzene.

Another key parameter in the coking operation is pressure which is controlled in a range of about 2 to about 5 bar (29 to 72 psi) to help manage the vapor-liquid equilibrium, influencing the yield of gases and lighter fractions versus solid coke.

The residence time within the coking operation is also controlled based on the desired products. Shorter residence times can promote the formation of lighter hydrocarbons, while longer times tend to produce more coke.

Furthermore, the composition of the plastic feedstock is important. Pre-treating the feedstock to remove impurities enhances the quality of the resulting base chemicals.

Adjusting the amount of heavy gas oil recycled back into the coking process (recycle ratio) can influence the production of lighter hydrocarbons.

The feedstock to the coking operation is typically preheated to around 350° C. to 400° C. (662° F. to 752° F.). Ensuring uniform preheating helps achieve consistent cracking reactions in the coker drum. The preheated feed is further heated to the desired cracking temperature in the coking furnace. Advanced control systems can be used to ensure precise temperature management.

The heated feed enters the coker drum where thermal cracking occurs, converting heavy molecules into lighter products and solid coke. The temperature and pressure within the drum are monitored and the feed rate and recycle ratio adjusted to optimize cracking.

Vaporized products 13 comprising vaporized light hydrocarbons from the coker unit 12 are separated into different fractions using fractionators. The temperature and pressure control in the fractionators ensure proper separation of gases, naphtha, diesel, and heavy gas oils.

Solid coke bottoms 14 is removed periodically from the coker drum of coker unit 12. Efficient decoking operations help to maintain continuous operation and prevent drum fouling.

In order to favor the production lighter olefins such as ethylene and propylene, coking operations are conducted at higher temperatures and short residence times. Fractionator settings are crucial to capture these gases effectively.

In order to favor the production of benzene, toluene, and xylene (BTX), coking operations are conducted at moderate temperatures and the specific feedstock compositions (aromatic-rich) is used to enhance the production of BTX compounds. Proper distillation and separation units are necessary.

In order to favor the production of light hydrocarbons, the coking operations are adjusted to optimize the cracking of heavy molecules into C₂-C₄ hydrocarbons.

In order to favor the production of coke, longer residence times and lower temperatures are utilized in the coking operations. Consistent drum temperature control and efficient decoking operations ensure high-quality coke output.

The process described herein may be advantageously practiced utilizing real-time sensors and analytics and Advanced Control Systems (APC) to monitor key parameters like temperature, pressure, and composition of the feed and products and to dynamically adjust operating conditions based on real-time data, ensuring optimal performance and product yield.

### Example

The following non-limiting example is provided to further illustrate the present invention.

### Crude Cracking and Fractionation of Lights

A prophetic example is provided to evaluate the efficiency of converting plastic waste into base chemicals in accordance with the present invention using a method analogous to crude oil cracking. This example outlines a hypothetical experimental setup designed to demonstrate the feasibility and efficiency of the process under specific conditions.

The waste plastic feedstock is mixed polyethylene (PE) and polypropylene (PP) plastic waste. The waste plastic feedstock is dissolved in a dissolution medium comprising xylene at 150° C., followed by catalytic decomposition using zeolite catalysts at 400° C. The dissolved plastic solution effluent is subjected to fractionation to separate lighter fractions such as ethylene and propylene using distillation columns. The heavy fraction from the distillation is processed in a delayed coker to produce aromatic compounds, olefins, specialty chemicals, and furfural.

The yields of this study is 55% ethylene, 45% propylene, and 15% C4+ hydrocarbons. The purity of the recovered base chemicals is comparable to those obtained from crude oil refining. The process demonstrates a 90% conversion efficiency, with minimal waste and by-products. Valuable by-products such as aromatics (benzene, toluene, xylene), olefins, specialty chemicals, and furfural are produced, enhancing the economic viability of the plastic recovery process.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above processes without departing from the scope of the invention, it is intended that all matter contained in the above description and shown in the accompanying drawings shall be interpreted as illustrative and not in a limiting sense.

What is claimed:

1. A process for recovery of base chemicals from plastic waste, comprising:
reducing the plastic waste to produce a particulate plastic waste;
dissolving the particulate plastic waste in a dissolution medium comprising an aromatic solvent to produce a dissolved plastic solution effluent;
fractionating the dissolved plastic solution effluent to produce an overheads fraction and a bottoms fraction;
catalytically decomposing the components of the bottom fraction to produce decomposition products; and
subjecting at least a portion of the decomposition products to a coking operation.

2. The process of claim 1, wherein the aromatic solvent is selected from the group consisting of toluene, xylene, benzene, styrene, ethylbenzene and mixtures thereof.

3. The process of claim 1, wherein the particulate plastic waste is dissolved in the dissolution medium at a temperature of from about 150° C. to about 300° C.

4. The process of claim 1, wherein the particulate plastic waste is dissolved in the dissolution medium in the presence of a catalyst.

5. The process of claim 4, wherein the particulate plastic waste is dissolved in the dissolution medium at a temperature of from about 100° C. to about 200° C.

6. The process of claim 1, further comprising recovering the aromatic solvent from the dissolved plastic solution effluent.

7. The process of claim 1, wherein the catalytic decomposition is conducted in the presence of a catalyst selected from the group consisting of zeolite catalysts, metal oxide catalysts, mixed metal catalysts and combinations thereof.

8. The process of claim 6, wherein the catalytic decomposition is conducted at a temperature of from about 300° C. to about 500° C.

9. The process of claim 1, wherein the coking operation is conducted in a delayed coker unit.

10. The process of claim 1, wherein the plastic waste comprises materials selected from the group consisting of high-density polyethylene (HDPE), low-density polyethylene (LDPE), linear low-density polyethylene (LLDPE), polypropylene (PP), polyethylene terephthalate (PET) and mixtures thereof.
